(19) United States
(12) Patent Application Publication
(10) Pub. No.: US 2009/0232280 A1
(43) Pub. Date: Sep. 17, 2009

(54) Film and storage plate protection systems and methods

(76) Inventors: Riccardo J. Tresso, Westminster, CO (US); Michael J. Vega, Longmont, CO (US)

Correspondence Address: LAW OFFICE OF CHAD C. SOLIZ , LLC, 407 N. Lincoln Ave. Ste. 105, LOVELAND, CO, 80537, US

(21) Appl. No.: 12/380,854

(22) Filed: Mar. 3, 2009

Related U.S. Application Data
(60) Provisional application No. 61/068,283, filed on Mar. 3, 2008.

(51) Int. Cl. A61B 6/14

(52) U.S. Cl. 378/169

(57) ABSTRACT

An intra-oral radiograph medium protection device in accordance with the present invention facilitates the comfort of the patient during radiology examination, while protecting radiograph medium such as film or storage plates from damage during the examination. Features of the present invention address protection of the radiograph medium during intra-oral manipulation and placement of the radiograph medium during examination, providing a fluid barrier, and variable positioning features, all while facilitating the ease of use of the radiograph medium in radiographic examination in preparation for, during and after the exam. The intra-oral radiograph medium protection device has two radiograph medium protection elements that are releasably retained in either open or closed configurations to provide a protective casing for a radiograph medium. Radiograph medium may be releasably retained within an interior of the two radiograph medium protection elements, the radiograph medium in preferred embodiments being retained within the two radiograph medium protection elements. The present invention relates and is directed to apparatus and methods of dentistry and dental examination. The present invention also provides processes and methods directed to one or more aspects of intra-oral radiological dental examination.

### BACKGROUND OF THE INVENTION

The present invention relates to systems for the protection of film and storage plate in dental examinations, such as traditional film and phosphor plate storage, and in some particular embodiments, to apparatus and methods of dentistry and dental examination. The present invention may also provide processes and methods directed to one or more aspects of dentistry and dental examination, such as to accommodate either film or phosphor plates, providing one or more of: protection of patient and equipment, fluid barrier, patient barrier, comfort, ease of use, reliable fluid barrier, and variable bite tab locations, as well as the use of x-ray and other examination equipment.

Reference to a patient is made throughout this specification. The reference may sometimes be made to human patients, for example, or alternatively reference can be made to a designation of an animal, such as in veterinary practice, or in testing environments upon live subjects, test equipment, and forensics, as but a few of the possible applications. In any event, each may be referred to generally as a patient and the present invention is applicable to the above applications and in the examination and care thereof as one of ordinary skill in the art would understand such technology.

Reference to an intra-oral x-ray or other examination is made throughout this specification. The reference may sometimes be made to other intra-oral examinations now known or later developed, for example any intra-oral examination, or other reference to a dental or veterinary practice, or in testing environments upon live or other testing equipment. In any event, each may be referred to generally as examination that is intra-oral or otherwise conducted within the oral cavity and the present invention is applicable to the above applications and in the examination and care thereof, including but not limited to x-ray technologies incorporating film or phosphor plates, as one of ordinary skill in the art would understand such technology.

Managing examination in dentistry environments can be a daunting undertaking that may require specialization and experience in not only dentistry but in various fields of dental assisting, hygiene, and examination processes, radiation and scanning science, as well as business practices. The ultimate objective may be the protection and adequate examination of the patient to achieve adequate examination and preferred care. However, the accommodation of either film or phosphor plates that may be used in traditional practice must be considered, while also providing for the reliable protection of this and other examination equipment, such as a fluid barrier for the equipment in light of the type of examination and technique as well as protection from external forces that may scratch or otherwise damage sensitive equipment and film and plates. Other factors must also be considered, such as the comfort of the patient, ease and efficient of use of examination equipment, and variable bite tab locations that may be required in traditional examination techniques. The everyday business operations and revenue of practice may in indeed be impacted by the type of equipment and methods chosen to accomplish effective examination practices.

Systems have been devised in the industry in an attempt to accommodate for both the traditional examination of patients utilizing film or phosphor plates for intra-oral x-ray of the patient while providing for some basic level of functionality in protecting the film or phosphor plate used and attempting to provide for an economical solution for everyday operations. The traditional technology may provide a combination of fluid barrier and positional alignment via plastic or vinyl bags or sheaths. These systems typically lack adequate protection of plates and film and may result in scratch or damaged x-ray medium. Other art provides basic hinged enclosures and fixed bite tabs that appears to require external equipment and positioning of the device. The concept of enclosures, covers or protectors for digital sensors and film and phosphor plate, as well as fixed bite tab technologies, have been disclosed in traditional technologies that may presently lack some or many the above-mentioned dental examination and business practice objectives.

Traditional technologies may suggest that there has been a heretofore identified but yet unanswered need for an inclusive intra-oral examination solution to achieve the above-stated objectives while providing for the everyday business operations and economic value necessary in order to maintain the traditional intra-oral examination as a viable business and medical practice. Heretofore known dentistry services, for example, may provide for and perform the function of intra-oral examination well, including the provision of some functionality in an x-ray examination solution, while leaving many of the objectives of the present invention lacking or unaddressed.

Heretofore known technologies may be directed to intra-oral examination using x-ray technologies; however there appear to be no current technologies that provide or even attempt to solve the above problems, address the identified needs, and that lack the functionality and inventive features of the present invention.

### SUMMARY OF THE INVENTION

The present invention relates to systems for protecting film and storage plate used for dental examination, such as traditional film and phosphor plate storage, and in some particular embodiments, to apparatus and methods of dentistry and dental examination. The present invention also provide processes and methods directed to one or more aspects of dentistry and intra-oral examination, such as to accommodate either film or phosphor plates, providing for the protection of patient and equipment, a reliable fluid barrier, a patient barrier, comfort of the patient, ease of use, and variable positioning features, as well as the use of x-ray and other examination equipment. The present invention is a relatively low cost item that may be provided in some embodiments as a disposable enclosure. An intra-oral radiograph medium protection device in accordance with the present invention facilitates the comfort of the patient during radiology examination, while protecting radiograph medium such as film or storage plates from damage during the examination.

Features of the present invention address protection of the radiograph medium during intra-oral manipulation and placement of the radiograph medium during examination, providing a fluid barrier, and variable positioning features, all while facilitating the ease of use of the radiograph medium in radiographic examination in preparation for, during and after the exam. The intra-oral radiograph medium protection device has two radiograph medium protection elements that are releasably retained in either open or closed configurations to provide a protective casing for a radiograph medium. Radiograph medium may be releasably retained within an interior of the two radiograph medium protection elements, the radiograph medium in preferred embodiments being entirely contained within the two radiograph medium protection elements. The present invention relates and is directed to apparatus and methods of dentistry and dental examination. The present invention also provides processes and methods directed to one or more aspects of intra-oral radiological dental examination.

Accordingly, in one embodiment, the invention is an intra-oral radiograph medium protection device, incorporating a first radiograph medium protection element having a fixed outer profile and a second radiograph medium protection element having a fixed outer profile, the first and said second radiograph medium protection elements may be releasably retained in either an open configuration or a closed configuration. A radiograph medium protection interior defined by the first and second sensor protection elements in the closed configuration accommodates radiograph medium, and a radiograph medium positioning element within the radiograph medium protection interior, positioning the radiograph medium within the sensor protection interior. The first and second radiograph medium protection elements releasably retain a radiograph medium within the sensor protection interior in the closed configuration and the first and second radiograph medium protection elements provide a fluid barrier about the extent of the radiograph medium.

In another embodiment, the invention is a method of protecting a radiograph medium during intra-oral examination, wherein featured steps are the positioning of a radiograph medium in an interior of a first radiograph medium protection element, connecting the first radiograph medium protection element with a second radiograph medium protection element, and releasably retaining the first and second radiograph medium protection elements in a closed configuration. Furthermore, a radiograph medium protection interior is defined by the first and second radiograph medium protection elements in the closed configuration, and the radiograph medium is releasably retained within the radiograph medium protection interior in the closed configuration. The radiograph medium is then positioned within the radiograph medium protection interior and a fluid barrier provided with the first and second radiograph medium protection elements about the extent of the radiograph medium.

In still further embodiments, the invention is a method of intra-oral examination, having the steps of providing a radiograph medium for intra-oral examination, positioning a radiograph medium in an interior of a first radiograph medium protection element, and connecting the first radiograph medium protection element with a second radiograph medium protection element, so as then to releasably retain the first and second radiograph medium protection elements in a closed configuration. Furthermore, the method provides defining a radiograph medium protection interior by the first and second radiograph medium protection elements in the closed configuration, so as to releasably retain the radiograph medium within the radiograph medium protection interior in the closed configuration. Radiograph medium is then positioned within the radiograph medium protection interior, and a fluid barrier is provided with the first and second radiograph medium protection elements about the extent of the radiograph medium. The radiograph medium is placed in an intra-oral location of a patient for intra-oral examination and examination of the patient is performed.

### BRIEF DESCRIPTION OF THE DRAWINGS

FIG. 1 is an isometric view of the interior of an embodiment of the present invention in an open configuration.

FIG. 2 is an isometric view of the exterior of the embodiment of FIG. 1 in an open configuration.

FIG. 3 is another isometric view of the embodiment of FIG. 1 in an open configuration and describing some features of the present invention in a different view.

FIG. 4 is an isometric view of the embodiment of FIG. 1 describing some features of the present invention in relation to a film element.

FIG. 5 is an isometric view of the exterior of the embodiment of FIG. 1 in an open configuration.

FIG. 6 is an isometric view of another embodiment of the present invention in an open configuration and described in relation to a phosphorus plate element.

FIG. 7 is an isometric view of the embodiment of FIG. 6 in an open configuration.

FIG. 8 is an isometric view of the embodiment of FIG. 6 in an open configuration and described in relation to a vinyl film element.

FIG. 9 is an isometric view of another embodiment of the present invention in an open configuration and described in relation to a vinyl film solution.

FIG. 10 are isometric views of some embodiments of the present invention.

FIG. 11 are isometric views of additional embodiments of the present invention.

FIG. 12 is a flow chart describing one embodiment of the present invention.

FIG. 13 is a flow chart describing an embodiment of the present invention.

### DETAILED DESCRIPTION OF PREFERRED EMBODIMENTS

The invention in some embodiments are processes and methods, as well as one or more apparatus and device, each embodiment providing functionality through either the apparatus or device or by way of the disclosed process and methods. The invention as previously described and as further described below is technology suitable for known and to be developed technologies incorporating various radiographic medium, such as traditional film and storage plate technologies. Those skilled in the art are familiar with traditional radiographic medium for intra-oral examination.

Furthermore, aspects of the invention may be described to be composed of preferred materials, while other embodiments can be provided in other materials as alternatives encompassed by the present invention as one skilled in the art would be familiar with the disclosed and equivalent materials. In preferred embodiments of the invention, the material has pliable characteristics that facilitate the objects of the present invention, but that also may add to the workability of the technology during examination procedures and may even add to the comfort of the patient. The present invention is also applicable to disposable embodiments that are preferentially designed to be a relatively low cost solution.

Embodiments of the invention may be now or later illustrated as a diagram of the functionality of the system or even as a configuration of an intra-oral examination system as but one embodiment of the present invention. The system may comprise the invention as well as known examination features and techniques. The invention may be described as a protection device used in some preferred embodiments for intra-oral examination and will preferably fit both standard size and other size variations of radiograph medium.

The invention may be described as a disposable barrier device or protection device used in some preferred embodiments for intra-oral x-rays and will fit both standard size traditional radiograph medium such as x-ray film and standard size storage plates such as phosphor storage plates. Although the disclosure may specifically reference standard size zero (0) film and plate, the technology and functionality are applicable to other sizes and for both x-ray film and phosphor storage plates and may be made to accommodate any size such as sizes 0 through 4 as understood by one skilled in the art. The disclosure is also applicable to disposable embodiments that are preferentially designed to be a relatively low cost solution.

In reference to traditional intra-oral examination, such techniques may require specific positioning of the radiograph medium and any holder in the mouth of the patient, primarily what is otherwise known as bite wing orientation and posterior, anterior and occlusal orientations. The present invention and its features seek to accommodate the traditional film and storage plate technologies and examination techniques in a novel approach to protecting the radiograph medium and in intra-oral examination, as well as in the introduction of the inventive devices herein.

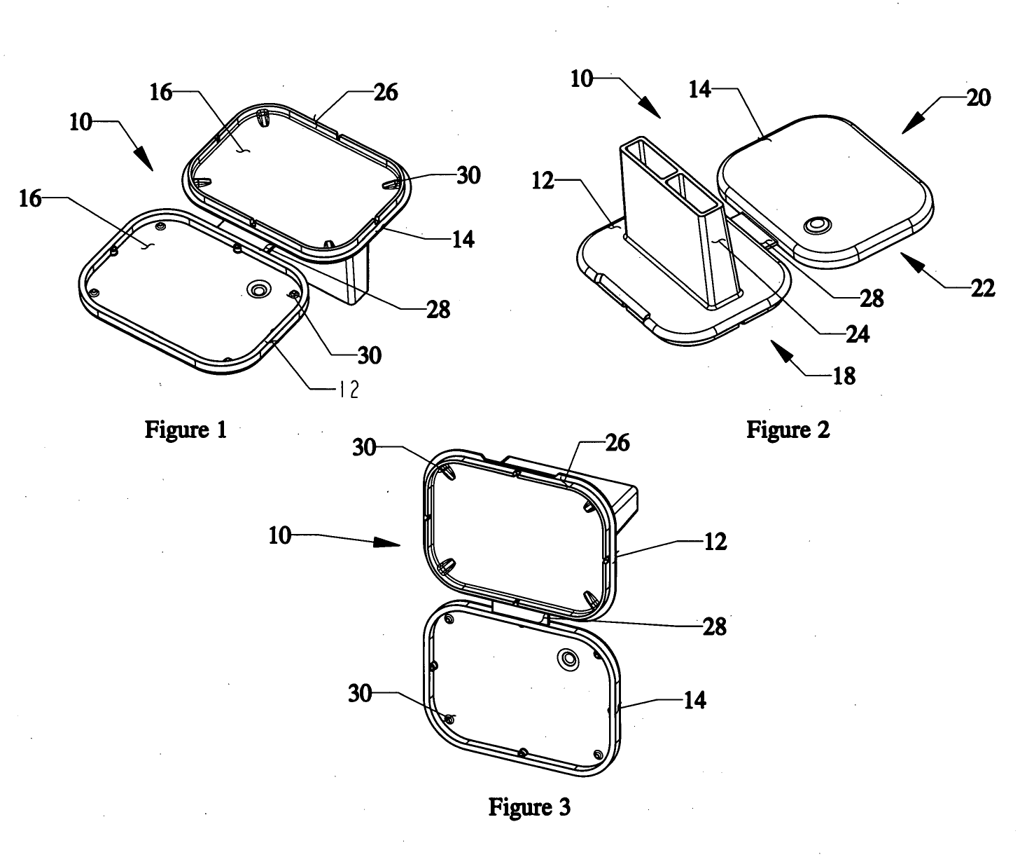

Accordingly, and in reference to FIG. 1, one embodiment of the invention is described as the intra-oral radiograph medium protection device 10. FIG. 1 describes the protection device 10 in an open configuration, wherein the first radiograph medium protection element 12 and second radiograph medium protection element 14 is shown in an open configuration. Each radiograph medium protection element has a fixed outer profile 18, 20, as described for example in FIG. 2. The outer profile can be provided in a number of shapes or contours, the outer profile having some relation and correspondence to the dictates of the size, shape and type of radiograph medium, such as the film or plate size, model or brand, and the type of application or examination technique to be used.

The radiograph medium protection element's 12, 14 outer profile 18, 20 are fixed in that the contour and shape of the profile are retained in the use of materials and the manufacture thereof to serve and to provide a physical barrier between the patient and the radiograph medium, particularly given the environment of intra-oral examination and a desire to retain a profile shape and contour to facilitate examination and repeated use, if desired. The invention encompasses varying configurations of protection devices, radiograph medium protection elements and fixed outer profiles as some alternative embodiments of the present invention.

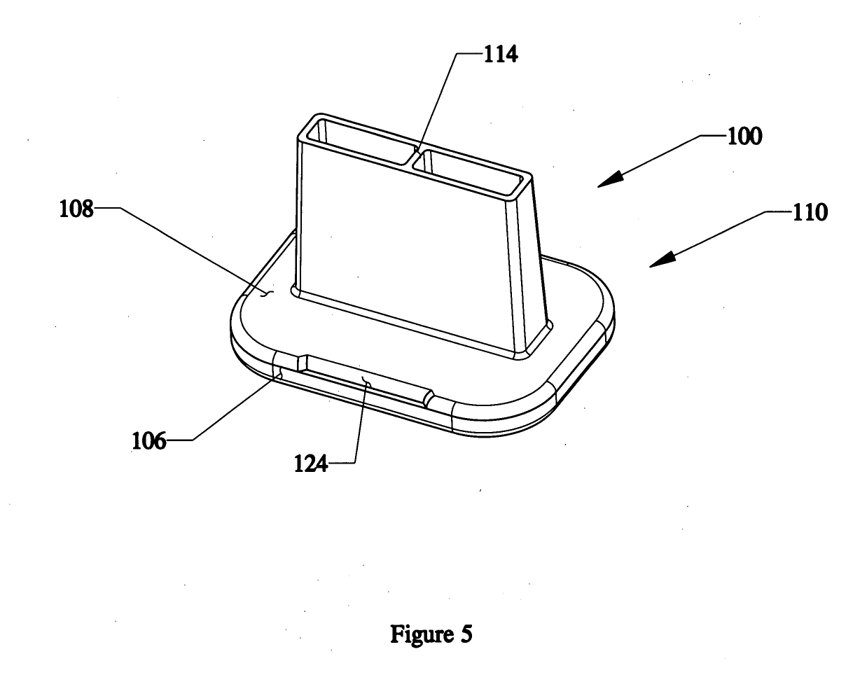

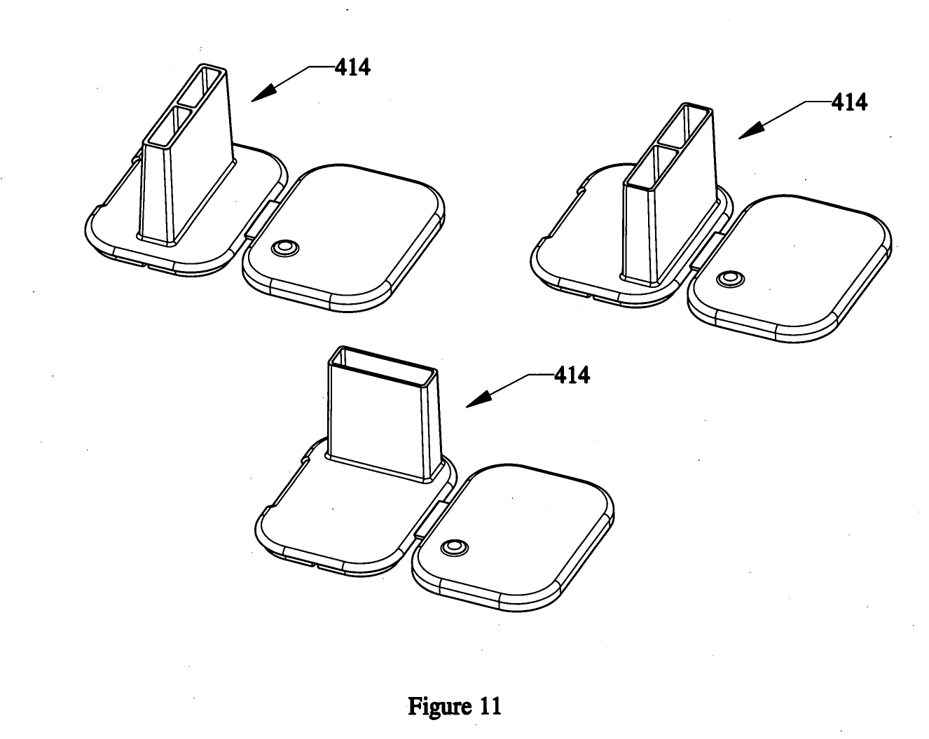

A radiograph medium protection interior 16 is shown in FIG. 1, an interior to each of the protection elements 12, 14. FIG. 2 describes device 10 in a rear view from which the radiograph medium protection interior 16 is not visible. The interior is defined by the first and second radiograph medium protection elements, and defined as a protection interior in a closed configuration of the protection elements (not shown in FIG. 1). FIGS. 5 and 10 as well as other disclosure herein describe a closed configuration of an intra-oral radiograph medium protection device 10.

Now in reference to FIGS. 1 and 5, the first and second radiograph medium protection elements 12, 14 may be retained in either the open or closed configurations, as also described in some other embodiments described in the figures. The open configuration may correspond prior to the placement of the radiograph medium within the intra-oral sensor protection device 10 or the removal of the radiograph medium, such as after examination. The closed configuration may correspond to the radiograph medium having been positioned within the radiograph medium protection interior 16 and, the first and second radiograph medium protection elements having been releasably retained in the closed configuration in order to protect the radiograph medium and the patient and to conduct examination.

As just described, the first and second radiograph medium protection elements 12, 14 releasably retain open and closed configuration in some preferred embodiments. The protection elements will remain open, or alternatively remain closed, and further features of the present invention as described herein facilitate the retention of the open and closed configurations, such as connection and lock features as well as hinge elements and the materials thereof. As the protection elements are retained in either the open or closed configurations, the protection device 10 may be released to provide for the alternative configuration, and in some embodiments, to provide movement of the first and second protection elements in protecting the radiograph medium and in conducting examination.

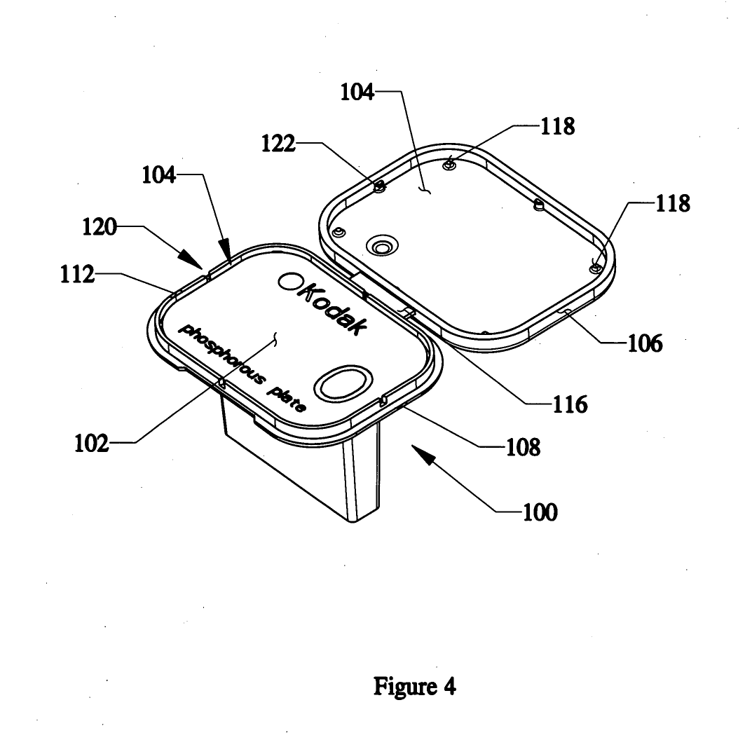

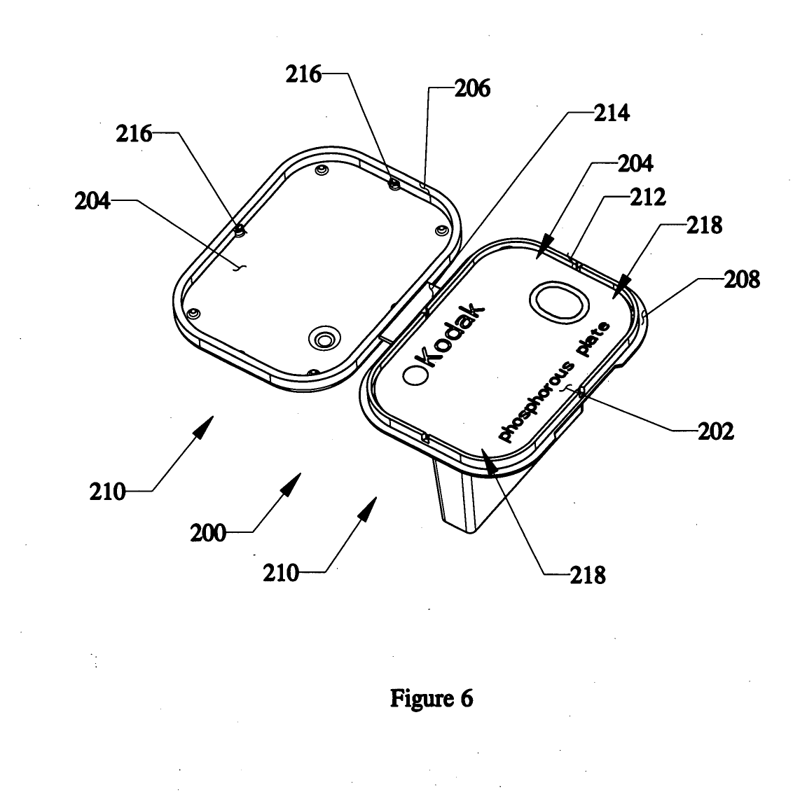

Accordingly, the first and second radiograph medium protection elements in some preferred embodiments releasably retain a radiograph medium that is positioned within the radiograph medium protection interior 16. In the open configuration a radiograph medium may be placed within the interior 16 and the first and second protection elements moved to a closed configuration (FIG. 5), thus defining the radiograph medium protection interior and retaining a radiograph medium placed within. Once configured in the closed configuration, the first and second radiograph medium protection elements provide a fluid barrier about the extent of the radiograph medium, as shown in FIGS. 5 and 10 as description of intra-oral radiograph medium protection device 100, 200. FIGS. 4 and 6 describe a traditional radiograph medium 102, 202 as a phosphor storage plate. Radiograph medium 102, 202 is positioned within the radiograph medium protection interior 104, 204 and protection elements 106, 108 and 206, 208 are releasably retained from an open to a closed configuration such that the radiograph medium 102, 202 is releasably retained within the radiograph medium protection interior 104, 204 and the first and second radiograph medium protection elements provide a fluid barrier about the extent of the radiograph medium.

In still further embodiments, the intra-oral radiograph medium protection devices 10, 100 and 200 as described in the figures may have a fixed profile radiograph medium casing 22, 110 and 210 defined by the fixed outer profiles of the first and second radiograph medium protection elements. The casing 22, 110 and 210 contain the extent of the radiograph medium within the radiograph medium protection interior and further provide a barrier about the extent of the radiograph medium. The casing 22, 110 and 210 also facilitate positioning intra-orally the protection devices 10, 100 and 200 as described below.

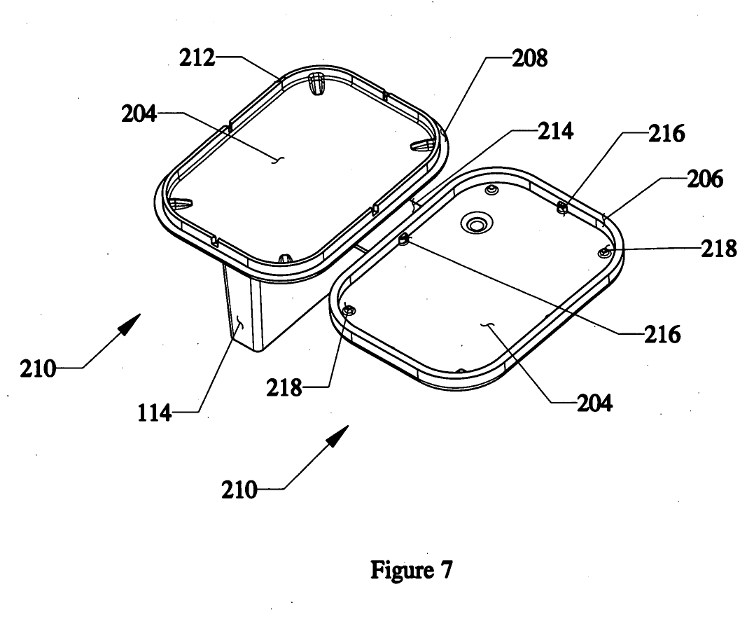

Now in reference to FIGS. 1 through 7 and the figures generally, the intra-oral radiograph medium protection device may have a radiograph medium casing positioning element 24, 114 and 214 of the radiograph medium casing that in some embodiments may be a feature of and unitary with one of the first and second sensor protection elements. Independent of the features of the present invention, mechanical elements of the positioning element may be referred to as a bite tab or a bite wing. As described in the figures, the position element may be a featured of one of the sensor protection elements and may be provided as a unitary feature and element of the radiograph medium protection element. FIGS. 10 and 11 describe a radiograph medium casing positioning element 314, 414 and its features in some embodiments. In some embodiments, and as shown in the Figures, the radiograph medium casing positioning element 24, 114 may be designed and configured such that configurations for bite wing, anterior and posterior of the radiograph medium protection device may be accomplished. FIGS. 10 and 11 describe the positioning elements in bitewing, posterior and anterior configuration, and even may be occusal in configuration.

Again, in reference to the figures, an intra-oral radiograph medium protection device can afford barrier protection, and in some embodiments, at least one or even both of the first and second radiograph medium protection elements have a radiograph medium protection connection element 26, 112 and 212. The radiograph medium protection connection element in preferred embodiments would be a connection between the first and second radiograph medium protection elements that may also serve to help provide the function of releasable retainment of the radiograph medium protection elements, and even provides a fluid barrier to the radiograph medium protection interior. In some preferred embodiments, a lap joint may be provided as the radiograph medium radiograph medium protection connection element and serve to connect the first and second radiograph medium protection elements. The mechanical joint is shown in the Figures; however, other connection means and barrier configurations may be utilized that would afford fluid barrier characteristics and that may also serve and provide the features described above. Barrier elements, such as the lap joint or joint barrier described in the figures, as well as hinge elements, all serve to afford fluid protection and releasable retainment of the radiograph medium protection elements and functionality of the intra-oral sensor protection device generally.

Some preferred embodiments provide features that further facilitate changing the open and closed configurations of the radiograph medium protection elements and in some embodiments, one or more hinge elements 28, 116 and 214 may be connected with the first and second radiograph medium protection elements. The first and second radiograph medium protection elements are releasably retained by the hinge element in either an open configuration or a closed configuration, and the hinge elements 28, 116 and 214 being connected with each of the radiograph medium protection elements, assists in essentially opening and closing the intra-oral radiograph medium protection device, being connected with the radiograph medium protection elements in providing hinge functionality.

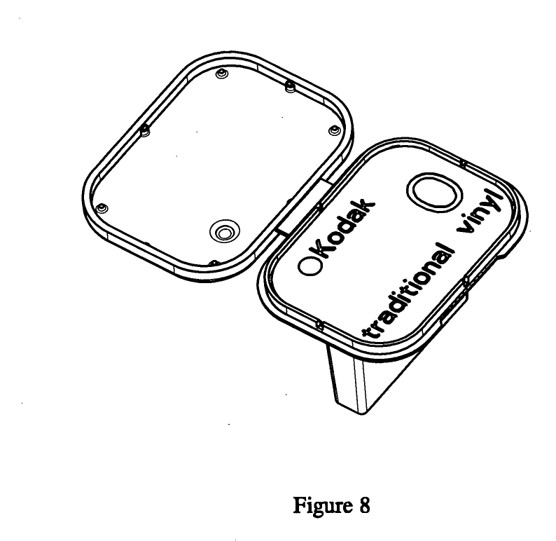

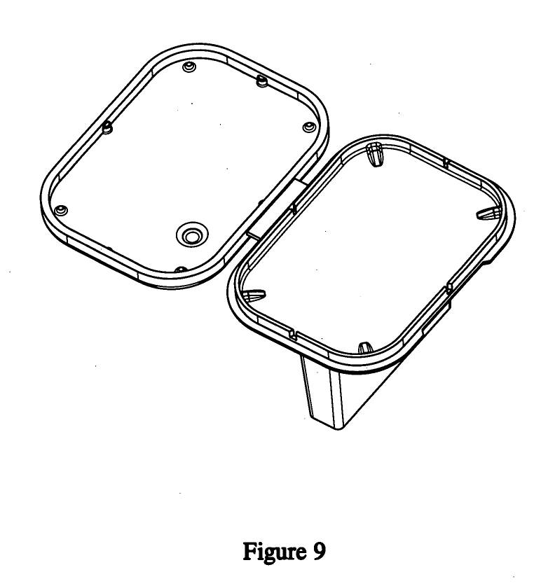

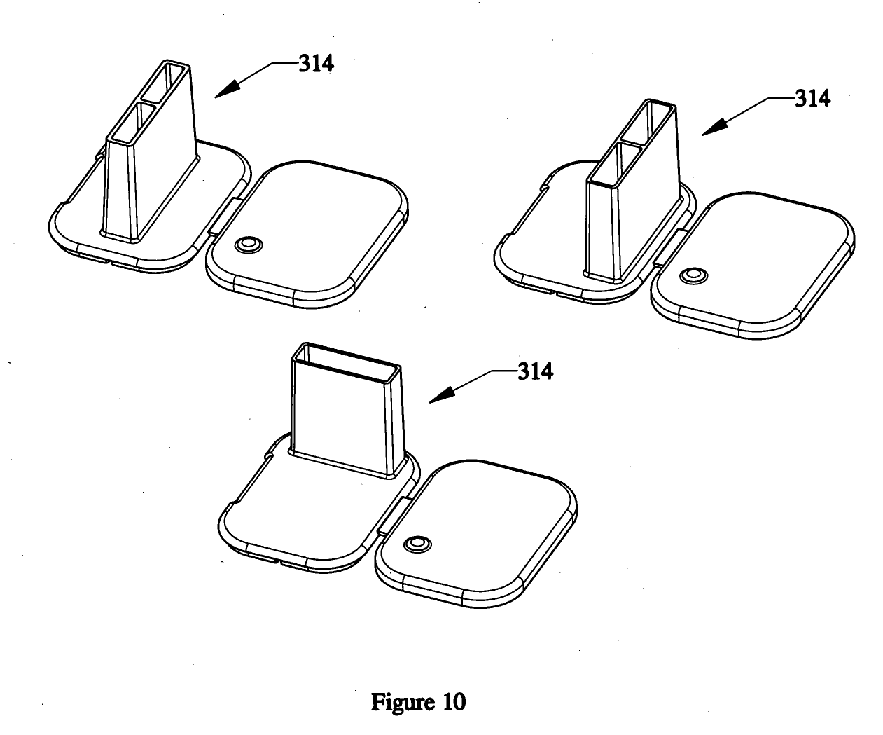

Now in reference to the figures and in some preferred embodiments, a radiograph medium positioning element or elements 30, 118 and 216 may be incorporated to adequately position the radiograph medium within the intra-oral radiograph medium protection device. In preferred embodiments, the radiograph medium positioning element is within the radiograph medium protection interior, the positioning element positioning the radiograph medium within the radiograph medium protection interior. Furthermore, and in some embodiments, locating and support elements, and in some embodiments locating and support pins 216, 218 may be provided in association with the first and second radiograph medium protection elements. FIGS. 8 and 9 describe embodiments of the invention having less positioning element features than other embodiments. The support and locating elements may be provided on the top, bottom, or combination of top and bottom within the radiograph medium protection interior, and in some embodiments such as those of the figures be the interior surface within the radiograph medium protection interior corresponding to the fixed outer profile, such as an internal shoulder accommodating the shape and contour of the sensor.

Additionally, in some preferred embodiments, an intra-oral radiograph medium sensor protection device can be provided with a locking element within the radiograph medium protection interior, the locking element releasably retaining the first and second radiograph medium protection elements in a closed configuration. In some embodiments, ridges, bumps, interference features or similar fastener elements may be incorporated to lock the first and second radiograph medium protection elements in a securely closed configuration while affording the opportunity to easily change to the open configuration. In one embodiment, interference features 120, 122 of FIG. 4 serve to mutually engage and securing a closed configuration. The provision of locking features therefore may assist in the securing the closing of the protection device. In some embodiments, these features may be referred to as interference features. In some particular embodiments, the locking elements may comprise corresponding location elements and locking elements. These features may not only provide for a removable lock for portions of the enclosure, but may also serve to provide a locating feature for examination media within the enclosure.

An opening element such as a detent, ridge, depression, notch, slot or similar mechanical feature may be provided exterior on the first or second or both radiograph medium protection elements, and preferably on the exterior of the fixed profile radiograph medium casing. FIG. 5 describes one such feature as a finger opening element 124. The finger opening element 124, and opening elements generally, provide a convenient and easy release of the first and second radiograph medium protection elements from a closed configuration to an open configuration by allowing the user to apply finger pressure to each radiograph medium protection element to comfortably force open the intra-oral radiograph medium protection device.

Again in reference to figures generally, the protection device may comprise bite wing or bite tab elements and further comprises locating features, and in some embodiments, locking features in association with the tab or wing. Accordingly, a bite tab or bitewing may be located and configured as a feature of the intra-oral radiograph medium protection device and in reference to any examination technique. Some features may provide location and configuration of the tab or wing at different locations depending upon the intra-oral examination techniques used and may adjustably lock into place. In some embodiments, the casing as previously described may have rounded or other configured corners and surfaces that in some embodiments address the comfort of the patient.

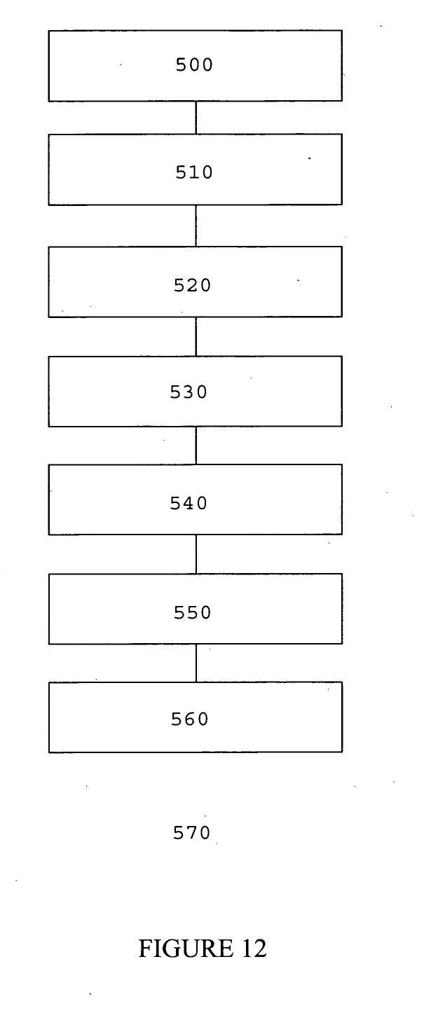

Furthermore, and in reference to FIG. 12, the present invention may be embodied as a method of protecting a radiograph medium during intra-oral examination. The features and functionality of the method and process embodiments correspond with the functionality previously described. Accordingly, in one embodiment, the method of protecting a radiograph medium during intra-oral examination may begin with positioning a radiograph medium in an interior of a first radiograph medium protection element 500 and connecting the first radiograph medium protection element with a second radiograph medium protection element 510. The first and second radiograph medium protection elements are then releasably retained in a closed configuration 520, and a radiograph medium protection interior is defined by the first and said second radiograph medium protection elements in the closed configuration 530. The radiograph medium may then be releasably retained within the radiograph medium protection interior in the closed configuration 540, and the radiograph medium positioned within the radiograph medium protection interior 550, and then providing a fluid barrier with the first and second radiograph medium protection elements about the extent of the radiograph medium 560. Further steps in accordance with some embodiments incorporate the step of providing a barrier between the radiograph medium and the patient with the first and second radiograph medium protection elements.

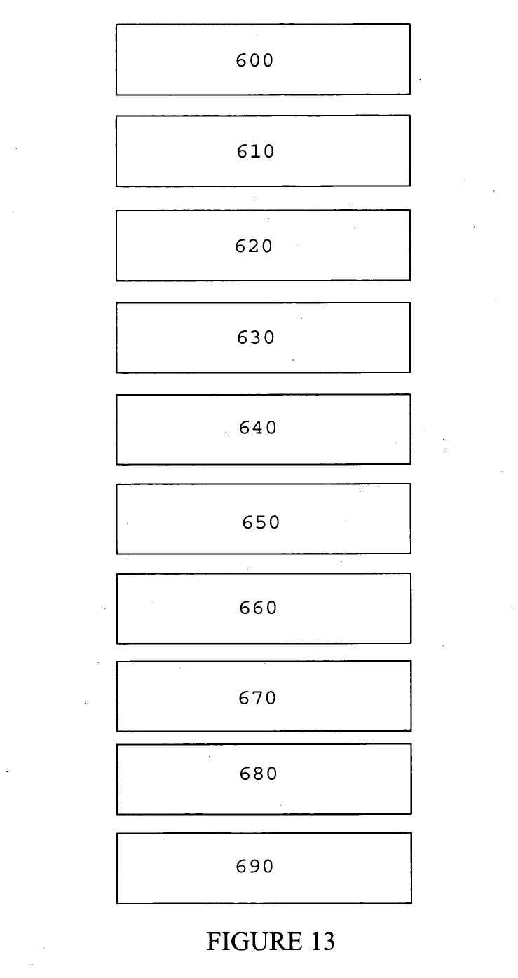

Furthermore, and now in reference to FIG. 13, the present invention may be embodied as a method of intra-oral examination. The features and functionality of the method and process embodiments correspond with the functionality previously described. Accordingly, in one embodiment, the method of intra-oral examination in accordance with the present invention may begin with providing a radiograph medium for intra-oral examination 600, a medium that may be selected based upon the requirements of the examination, and positioning a radiograph medium in an interior of a first radiograph medium protection element 610. These steps may be followed by connecting the first radiograph medium protection element with a second radiograph medium protection element 620, releasably retaining the first and second radiograph medium protection elements in a closed configuration 630, and, defining a radiograph medium protection interior by the first and second radiograph medium protection elements in the closed configuration 640. The method embodiment may continue with the step of releasably retaining the radiograph medium within the radiograph medium protection interior in the closed configuration 650, positioning the radiograph medium within the radiograph medium protection interior 660, providing a fluid barrier with the first and second radiograph medium protection elements about the extent of the sensor 670, placing the radiograph medium in an intra-oral location of a patient for intra-oral examination 680, and performing examination of the patient 690.

The embodiment of FIG. 13 may further incorporate additional steps. In some embodiments, providing a barrier between the radiograph medium and the patient with the first and second radiograph medium protection is performed. Also, the step of positioning a radiograph medium casing with a radiograph medium positioning element of the first and second radiograph medium protection elements may be performed. Steps consistent with the method of the present invention further provide in some embodiments: the step of positioning a radiograph medium casing with a radiograph medium positioning element comprises positioning a radiograph medium casing in a bite wing configuration; the step of positioning a radiograph medium casing with a radiograph medium positioning element comprises positioning a radiograph medium casing in a posterior configuration; the step of positioning a radiograph medium casing with a radiograph medium positioning element comprises positioning a radiograph medium casing in an anterior configuration; and the step of positioning a radiograph medium casing with a radiograph medium positioning element comprises positioning a radiograph medium casing in an occlusal configuration.

As can be easily understood from the foregoing, the basic concepts of the present invention may be embodied in a variety of ways. It involves one or more apparatus, device, processes, and business methods of dentistry and intra-oral examination, and in some particular embodiments, to methods of business, and apparatus, devices, processes and methods directed to one or more patients, as well as the use of or incorporation of traditional technologies, as well as devices, apparatus, singular or plural, and assemblies to accomplish the appropriate function. In this application, the techniques are disclosed as part of the functionality of the various devices described and as steps that may be inherent to utilization. In addition, while some devices are disclosed, it should be understood that these not only accomplish certain methods but also can be varied in a number of ways. Importantly, as to all of the foregoing, all of these facets should be understood to be encompassed by this disclosure.

Any acts of law, statutes, regulations, or rules mentioned in this application for patent; or patents, publications, or other references mentioned in this application for patent are hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference.

Further, if or when used, the use of the transitional phrase “comprising” is used “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

We claim:

1. An intra-oral radiograph medium protection device, comprising:
a first radiograph medium protection element having a fixed outer profile;
a second radiograph medium protection element having a fixed outer profile, wherein said first and said second radiograph medium protection elements may be releasably retained in either an open configuration or a closed configuration;
a radiograph medium protection interior defined by said first and said second sensor protection elements in said closed configuration; and
a radiograph medium positioning element within said radiograph medium protection interior, said radiograph medium positioning element positioning said radiograph medium within said sensor protection interior;
wherein said first and second radiograph medium protection elements releasably retain a radiograph medium within said radiograph medium protection interior in said closed configuration and wherein said first and second radiograph medium protection elements provide a fluid barrier about the extent of said radiograph medium.

2. An intra-oral radiograph medium protection device as described in claim 1, wherein said radiograph medium comprises a film element.

3. An intra-oral radiograph medium protection device as described in claim 1, wherein said radiograph medium comprises a storage plate.

4. An intra-oral radiograph medium protection device as described in claim 1, further comprising a fixed profile radiograph medium casing defined by said fixed outer profiles, said fixed profile radiograph medium casing containing the extent of said radiograph medium within said radiograph medium protection interior and providing a barrier about the extent of said radiograph medium.

5. An intra-oral radiograph medium protection device as described in claim 1, wherein at least one of said first and second radiograph medium protection elements comprise a radiograph medium protection connection element.

6. An intra-oral radiograph medium protection device as described in claim 5, wherein said radiograph medium protection connection element comprises a connection releasably retaining said first and second radiograph medium protection elements.

7. An intra-oral radiograph medium protection device as described in claim 6, wherein said radiograph medium protection connection element provides a fluid barrier to said radiograph medium protection interior.

8. An intra-oral radiograph medium protection device as described in claim 7, wherein said radiograph medium protection connection element comprises a lap joint.

9. An intra-oral radiograph medium protection device as described in claim 1, further comprising a hinge element connected with said first and second radiograph medium protection elements, wherein said first and second radiograph medium protection elements may be releasably retained by said hinge element in either an open configuration or a closed configuration.

10. An intra-oral radiograph medium protection device as described in claim 1, wherein said a radiograph medium positioning element comprises a locking element.

11. An intra-oral radiograph medium protection device as described in claim 10, wherein said locking element comprises a plurality of pins wherein each of said plurality of pins is configured for interfering locking engagement with one other of said plurality of pins.

12. An intra-oral radiograph medium protection device as described in claim 10, wherein said plurality of pins are unitary with one of said first and second radiograph medium protection elements.

13. An intra-oral radiograph medium protection device as described in claim 1, wherein said radiograph medium positioning element comprises a plurality of radiograph medium positioning elements within said radiograph medium protection interior.

14. An intra-oral radiograph medium protection device as described in claim 4, wherein one of said first and second radiograph medium protection elements comprises a radiograph medium casing positioning element.

15. An intra-oral radiograph medium protection device as described in claim 4, wherein said radiograph medium casing positioning element comprises a positioning element selected from the group consisting of: an anterior positioning element, a posterior positioning element, a bite wing positing element; and a occlusal positioning element.

16. An intra-oral radiograph medium protection device as described in claim 1, further comprising a locking element within said radiograph medium protection interior, said locking element releasably retaining said first and second radiograph medium protection elements in a closed configuration.

17. An intra-oral radiograph medium protection device as described in claim 16, wherein said locking element comprises a plurality of pins wherein each of said plurality of pins is configured for interfering locking engagement with one other of said plurality of pins.

18. An intra-oral radiograph medium protection device as described in claim 4, further comprising an opening element on the exterior of said fixed profile radiograph medium casing, said opening element providing release of said first and second radiograph medium protection elements from a closed configuration to an open configuration.

19. A method of protecting a radiograph medium during intra-oral examination, comprising the steps of:
positioning a radiograph medium in an interior of a first radiograph medium protection element;
connecting said first radiograph medium protection element with a second radiograph medium protection element;
releasably retaining said first and second radiograph medium protection elements in a closed configuration;
defining a radiograph medium protection interior by said first and said second radiograph medium protection elements in said closed configuration;
releasably retaining said radiograph medium within said radiograph medium protection interior in said closed configuration;
positioning said radiograph medium within said radiograph medium protection interior; and
providing a fluid barrier with said first and second radiograph medium protection elements about the extent of said radiograph medium.

20. A method of protecting a radiograph medium during intra-oral examination as described in claim 19, further comprising the step of providing a barrier between the radiograph medium and the patient with said first and second radiograph medium protection elements.

21. A method of intra-oral examination, comprising the steps of:
providing a radiograph medium for intra-oral examination;
positioning a radiograph medium in an interior of a first radiograph medium protection element;
connecting said first radiograph medium protection element with a second radiograph medium protection element;
releasably retaining said first and second radiograph medium protection elements in a closed configuration;
defining a radiograph medium protection interior by said first and said second radiograph medium protection elements in said closed configuration;
releasably retaining said radiograph medium within said radiograph medium protection interior in said closed configuration;
positioning said radiograph medium within said radiograph medium protection interior;
providing a fluid barrier with said first and second radiograph medium protection elements about the extent of said sensor;
placing said radiograph medium in an intra-oral location of a patient for intra-oral examination; and
performing examination of the patient.

22. A method of intra-oral examination as described in claim 21, further comprising the step of providing a barrier between the radiograph medium and the patient with said first and second radiograph medium protection.

23. A method of intra-oral examination as described in claim 21, further comprising the step of positioning a radiograph medium casing with a radiograph medium positioning element of said first and second radiograph medium protection elements.

24. A method of intra-oral examination as described in claim 23, wherein said step of positioning a radiograph medium casing with a radiograph medium positioning element comprises positioning a radiograph medium casing in a bite wing configuration.

25. A method of intra-oral examination as described in claim 23, wherein said step of positioning a radiograph medium casing with a radiograph medium positioning element comprises positioning a radiograph medium casing in a posterior configuration.

26. A method of intra-oral examination as described in claim 23, wherein said step of positioning a radiograph medium casing with a radiograph medium positioning element comprises positioning a radiograph medium casing in an anterior configuration.

27. A method of intra-oral examination as described in claim 23, wherein said step of positioning a radiograph medium casing with a radiograph medium positioning element comprises positioning a radiograph medium casing in an occlusal configuration.
